(19) United States
(12) Patent Application Publication
(10) Pub. No.: US 2022/0276188 A1
(43) Pub. Date: Sep. 1, 2022

(54) DEVICE AND METHOD FOR SIMULTANEOUSLY DETERMINING TEMPERATURE-DEPENDENT THERMAL CONDUCTIVITY, THERMAL DIFFUSIVITY AND SPECIFIC HEAT CAPACITY

(71) Applicant: Friedrich-Schiller-Universitaet Jena, Jena (DE)

(72) Inventors: Markus RETTENMAYR, Jena (DE); Stephanie LIPPMANN, Jena (DE); Anton ZIMARE, Jena (DE)

(21) Appl. No.: 17/425,544

(22) PCT Filed: Jan. 24, 2020
(86) PCT No.: PCT/DE2020/000010

§ 371 Date: May 13, 2022

(30) Foreign Application Priority Data
Jan. 24, 2019 (DE) 10 2019 00 590.2

(51) Int. Cl. G01N 25/18; G01N 25/00; G01N 25/20; G01J 5/12

(52) U.S. Cl. CPC G01N 25/18; G01N 25/005; G01N 25/20; G01J 5/12

(57) ABSTRACT

The invention relates to a device and a method for simultaneously determining temperature-dependent thermal conductivity, thermal diffusivity and specific heat capacity and comprises a heat source for locally heating a solid body to be examined, a both locally and chronologically high-resolution line and/or surface detector for non-contact temperature measurement along the sample, and a cooling Circuit having a cooling liquid flowing around the lower sample edge, the temperature increase and flow rate of which cooling liquid are measured continuously. The thermal diffusivity is determined by means of the described method from the transient thermal States of the sample, which are adjusted in a controlled manner, during heating and cooling. The thermal conductivity is determined from the steady state with a constant heating output. The specific heat capacity of the sample material is calculated according to the temperature from the data sets relating to the thermal diffusivity and thermal conductivity, which data sets are determined directly and over a large temperature range. Because of the enormous savings in time as compared with the prior art, a large number of different solid bodies can be comprehensively characterized thermally for the first time by means of the invention.

The invention relates to a method for simultaneously determining temperature-dependent thermal conductivity, thermal diffusivity and specific heat capacity having the features of claim 1, and to a device therefor having the features of claim 10.

Thermal material properties, such as thermal conductivity, thermal diffusivity or specific heat capacity, are very important in basic research and for simulation calculations with materials science-related problems and also for industrial manufacturing processes and applications. Metallurgical methods, the development of new components and technical processes under thermal load or the development of new materials, such as, e.g., thermoelectric materials, are substantially dependent on the knowledge of the respective thermal properties of the materials used. Therefore, there is a specific scientific and economic requirement for thermal material properties in a multitude of industrial fields of application.

From the prior art, there are known different devices and methods for determining the three central temperature-dependent material properties: thermal conductivity, thermal diffusivity and specific heat capacity.

Presently, the temperature-dependent thermal conductivity can be determined from steady-state temperature profiles along a sample to be examined. For this purpose, one end of the sample to be examined is brought into contact with a reference sample having known thermal properties. Then, one end is heated and the temperature profile of both samples is determined along the common sample axis with the aid of thermocouples. Using the knowledge of the temperatures at the measuring points and the distance between said points, the temperature-dependent thermal conductivity can be derived from the deviation of the measurement curve from a linear progression and the ratio of the gradients of the temperature profiles of the sample to be examined and of the reference sample.

The local resolution of this method is limited, whereby small temperature changes along the sample axis are barely detectable, or can only be detected with insufficient accuracy. Furthermore, a precise temperature measurement is only ensured when the thermocouples are positioned or welded within the sample. Owing to the contacting temperature measurement, thermal bridges to the surrounding area are produced, whereby the accuracy of these measuring methods is limited. The small local resolution and the contacting and inaccurate temperature measurement are both disadvantageous in these methods.

Furthermore, owing to the thermal inertia of thermocouples, it is not possible to detect rapid and small changes in the temperature profile of the sample material in a sufficiently precise manner.

A further disadvantage of the method described above resides in the dependency of the measurement result on the quality of the thermal contact between the two sample rods. By using special thermally conductive paste, the heat transmission to the end faces of the two sample rods can be improved but, owing to the thermal properties of the respective thermally conductive paste, an undefined heat transfer is generated which influences the measurement result. Furthermore, the examinable temperature range is greatly limited by the thermal stability of the reference sample and thermally conductive paste used.

In a modification of the above-described method, the temperature is measured using a pyrometer. An average is taken of the detected radiation intensity over a relatively large measurement spot. Owing to the limited local resolution in this method, local temperature changes can only be detected in a very limited manner and abrupt changes in the thermal properties, as occur, e.g., in the case of magnetic transitions, cannot be detected.

Furthermore, by using the above-described method it is not possible to determine thermal diffusivity or specific heat capacity.

A further method for determining thermal diffusivity which is known as LFM (laser flash method) and is patented (U.S. Pat. No. 4,928,254 A) uses the transient heating of a sample to be examined, which heating is generated using a laser pulse.

The sample material to be examined is placed in the measuring device on a holder having at least three webs, is heated to a predetermined temperature and is kept at that temperature until thermal equilibrium is achieved. A short laser pulse coupled-in via a lens on one side of the mostly circular, blackened sample disc results in a temperature increase on the opposite side of the sample disc. Using the knowledge of the laser power used and the recorded temperature change over time on the opposite sample side, the thermal diffusivity can be calculated for the predetermined temperature.

The defined coupling-in of laser radiation is significantly dependent on the condition of the irradiated surface. The sample must be uniformly blackened and have a defined roughness. Furthermore, the sample disc must have plane-parallel contact surfaces for reliable evaluation. A decisive disadvantage of a device using the LFM method is the creation of thermal bridges over the contact points of the sample disc and the distortion of the temperature measurement associated therewith.

A modification of the original LFM is proposed in DE 43 01 987 A1. An attempt is made therein to quantify the natural losses when coupling electromagnetic radiation into an absorbing sample and to incorporate these in the calculation of the thermal material properties. However, the compensation is always related to a comparison with a reference sample having known optical and thermal properties. Therefore, this modification of the original LFM cannot be effected without previous calibration. Furthermore, the reflection radiation sensor used therein must be adapted to the wavelength range of the pulse radiation source. Therefore, in order to generate the optical excitation, only ever one radiation source adjusted to the rest of the apparatus can be used. The direct consequence is either a limitation of the sample materials to be examined owing to their optical properties or additional outlay in terms of machinery or costs in order to ensure the use of the constantly optimized measuring apparatus. Furthermore, the accuracy when determining the natural losses when coupling-in of electromagnetic radiation increases in this case with the comparability of sample and reference, which requires additional outlay in the preparatory phase.

Other patents based on LFM, such as DE 10 2012 106 955 B4 or DE 10 2015 118 856 B3, are conceptual improvements over the original LFM, but still cannot be performed without corresponding reference measurements in order, e.g., to determine the specific heat capacity of the sample material to be examined, and thus to infer thermal conductivity in conjunction with the actual measurement result, the thermal diffusivity. However, the time disadvantage by successively scanning a comparatively large, practically relevant temperature range is not remedied thereby. The disadvantage of undesired heat dissipation over the contact points of the sample holder and the thereby distorted temperature measurement still remains.

Other methods, as described for example in DE 34 25 561 C2, measure the temperature increase of a sample heated with electromagnetic radiation with the aid of measuring sensors which are pressed against the sample surface using correspondingly dimensioned springs. These sensors generate a defined voltage drop, depending on the temperature, which is used in this case as a measurement variable. It is assumed that the conversion of the measured voltage into an equivalent temperature is linear. However, in practice this is only valid in a correspondingly small temperature range. Furthermore, the precision and accuracy of the measurement is adversely affected by the signal-to-noise ratio which varies from measuring sensor to measuring sensor and deteriorates along the length of the sensors. Furthermore, the measuring sensors on the sample and also the springs form thermal bridges which distort the measurement result.

In order to determine—in addition to the thermal diffusivity of the sample material to be examined—the thermal conductivity thereof, it is necessary to determine the specific heat capacity using another device (e.g., by means of “differential scanning calorimetry” (DSC); U.S. Pat. No. 3,263,484 A).

When determining specific heat capacity, the DSC uses the comparison of heat flows which flow away or towards the sample to be examined or measures the heating output required for the temperature change. The measured values are compared with a reference sample having known thermal properties.

Since the DSC always uses a comparison with a material having known thermal properties, this method cannot be performed without calibration by a trained person skilled in the art. Therefore, the accuracy of this method is always dependent on the experimental skill of the experimenter and the quality of the reference measurement.

Both LFM and also DSC have the disadvantage that a reliable determination of thermal diffusivity or specific heat capacity of the sample material to be examined is possible in each case only at a given temperature. Therefore, the entire measurement process must be repeated step-by-step for different temperatures. In order to be able to characterize a practically relevant temperature range of room temperature to (depending upon the material, considerably higher than) 0=1000° C. in sufficiently small steps, measuring times of tens of hours to days are required. If there is thus the requirement for temperature-dependent determination of the thermal diffusivity, thermal conductivity and heat capacity of a material, then the respective measuring times add up and rapid characterization is not possible.

A further disadvantage is that only thermal diffusivity can be determined using LFM and only specific heat capacity can be determined using DSC. Thermal conductivity cannot be measured directly using these two methods.

If thermal conductivity is determined from the two mutually independently obtained data sets of the LFM measurement and the DSC measurement, it should be taken into account that the respective errors in the individual measuring methods also add up when determining thermal conductivity.

A method known from patent DE 10 2008 012 758 B4 uses, similarly to DSC, a temperature-stable environment and infers, from the temperature changes initiated in a sample, the thermal properties thereof, such as, e.g., the thermal diffusivity or transformation heat of solid materials. The temperature change of the sample is initiated by the sample being transferred from a temperature-stable environment to an environment with a different temperature. A thermocouple provided at a defined position within the sample measures the change over time of the sample temperature, from which the thermal diffusivity can be calculated with precise knowledge of the sample geometry. The thermocouple thus has a large influence on the accuracy of the measurement because it also forms a thermal bridge to the surrounding area at this location and causes undesired admission or discharge of heat. Furthermore, heat losses owing to radiation from the sample surface are disregarded, whereby the measurement result is decisively distorted in particular at higher temperatures. It is also very disadvantageous that the measurement always relates only to one temperature and the determination of the temperature dependency of the thermal properties is not possible with a practicable amount of time.

A further method for determining thermal diffusivity, thermal conductivity and specific heat capacity known from patent DE 199 43 076 C2 heats a cylindrical sample at a predetermined heating rate in a controlled manner in a resistance-heated adiabatic heating system and records the heating output required for heating. The temperature of the sample is measured at the center of the sample and at the edge. The measurements are taken in a vacuum or in an inert atmosphere. This method is disadvantageous in that the heating output occurs via the measuring of the heating voltage at a defined point within the heating system. However, the electrical resistance and as a result also the decreasing voltage naturally also change with the temperature. Therefore, a precise determination of the heating output is not possible without knowledge of the temperature-dependent resistance of the heating wire. Furthermore, in order to ensure the required adiabatic ratios, a complex structure and complicated control of mutually independently mounted temperature control circuits is required. Furthermore, temperatures sensors positioned in bores within the sample and at the edge of the sample are used for determining the thermal properties. The temperature measurement is distorted in this case either by thermal bridges to the surrounding area or by the lack of direct contact with the sample. Another disadvantage is the fact that precise determination of the specific heat capacity of the sample is only possible with knowledge of the specific heat capacity of the entire measuring system and as a result by calculating the difference. It is thus necessary to perform a careful and time-consuming calibration prior to each measurement. The thermal properties determined using a measuring process are each valid for only one temperature. In order to ascertain the temperature-dependency of the thermal properties, many individual measurements and many calibration measurements are necessary, both of which increase the amount of time required and render a complete thermal characterization impracticable.

A further development of the method described in patent DE 199 43 076 C2 is found in patent DE 10 2004 051 875 B4. That patent describes an absolute method for simultaneously determining thermal diffusivity, thermal conductivity, specific heat capacity, transformation heat and sample density of a solid cylindrical sample. The improvement over patent DE 199 43 076 C2 resides in an adapted sample geometry which facilitates temperature measurement and adaptation. The above-mentioned limitations of the method, such as the temperature-dependency of the voltage drop when measuring the heating output, the distortion of the temperature measurement by thermal bridges or the lack of contact and the large amount of time required owing to the determination of the thermal properties at only one temperature in each case, are still present.

According to the previous prior art, there is no device and no method which can be used to determine thermal conductivity, thermal diffusivity and specific heat capacity directly, in a temperature-dependent manner and simultaneously over a large temperature range in one measuring cycle using one device.

The object of the present invention is to provide a method for simultaneously determining thermal conductivity, thermal diffusivity and specific heat capacity of a sample with the aid of an integrated measuring apparatus which is characterized by a rapid and reliable determination of the desired measurement variables in a single measuring cycle and without previous calibration. In the process, thermal bridges which lead to undesired heat flows and distort the temperature measurement are excluded by a non-contact temperature measurement.

In particular, a device and a method are to be described, with which it is possible to determine the three central material properties of thermal conductivity, thermal diffusivity and specific heat capacity simultaneously.

The invention relates to a device and a method for simultaneously determining temperature-dependent thermal conductivity, thermal diffusivity and specific heat capacity and comprises a heat source for locally heating a solid body to be examined, a both locally and chronologically high-resolution line and/or surface detector for non-contact temperature measurement along the sample, and a cooling circuit having a cooling liquid flowing around the lower sample edge, the temperature increase and flow rate of which cooling liquid are measured continuously. From the transient, thermal states of the sample, which are set in a controlled manner, during heating and cooling, the thermal diffusivity is determined by the method described. Thermal conductivity is determined from the steady state at constant heating output. From the data sets of thermal diffusivity and thermal conductivity which are determined directly and over a large temperature range, the specific heat capacity of the sample material is calculated in a temperature-dependent manner. By reason of the enormous time savings compared to the prior art, a large number of different solid bodies can be thermally characterized comprehensively for the first time with the aid of the invention.

Here, thermal conductivity, thermal diffusivity and specific heat capacity are determined directly with a single device.

The method described in this case is a direct method for determining absolute values which does not require calibration.

Furthermore, the device described in this case can be used to characterize these three thermal material properties directly and over a large temperature range. This results in an enormous time saving compared with the prior art. In order to cover, e.g., a practically relevant temperature range of ϑ=20 . . . 1000° C., the simultaneous and temperature-dependent determination of thermal diffusivity, thermal conductivity and specific heat capacity within one measuring cycle serves to reduce the time required to a few hours or minutes compared to the prior art. Therefore, the time and economic savings mean that a comprehensive thermal characterization of a wide variety of materials is made possible in the first place.

The objects formulated here are achieved by a method which uses thermally transient and steady states of a sample to be examined in order to determine the three central thermal material properties of thermal diffusivity, thermal conductivity and specific heat capacity, according to claim 1, and by a device for transferring the sample to these thermal states as a result of a quasi-one-dimensional heat flow through this sample, characterized by claim 10. Expedient or advantageous embodiments of the device and of the method are formulated in the corresponding dependent claims.

The device for simultaneously determining temperature-dependent thermal conductivity, thermal diffusivity and specific heat capacity comprises the one-sided, controlled heating of a sample to be examined.

One embodiment of the device in accordance with the invention comprises an induction furnace, with which it is possible to heat electrically conductive materials in a contactless manner, by means of eddy currents by reason of inductive coupling of a medium- or high-frequency alternating field, generated by an induction coil.

In one modification of said embodiment, an electrically non-conductive or semi-conductive sample material is heated on one side with the aid of a susceptor. Here, the eddy currents are only generated in the susceptor and the sample heats up via thermal conduction by reason of direct thermal contact with the susceptor. The thermal contact between the susceptor and the heated sample end is not disadvantageous for determining thermal diffusivity and thermal conductivity, since the heat flow through the sample remains one-dimensional and is measured directly. Therefore, there are no disadvantages with regard to the use of a susceptor compared to other embodiments. Thus, with the aid of a susceptor, the device in accordance with the invention can be used to characterize electrically conductive, semi-conductive and electrically non-conductive materials with regard to their thermal material properties.

In a further embodiment, the one-sided heating of the sample is effected in a contactless manner with the aid of a laser by coupling in electromagnetic beams. Modern fiber lasers, for example, allow controlled emission of the radiation or heating output and thus, in combination with non-contact temperature measurement, likewise open up the possibility of precisely controlled heating of the sample. One advantage of using a laser as a heating source is the possibility of heating the sample independently of the electrical properties of the examined sample material (conductive, semi-conductive or non-conductive).

In another embodiment, the sample is heated on one side with a resistance-heated heating system. An optically measured temperature is used to control the heating system. As also in the case of the embodiments with the susceptor and laser, a resistance-heated heating system offers the possibility of thermally characterizing electrically conductive, semi-conductive and non-conductive materials.

The temperature of the heated sample end is likewise measured and controlled in a contactless manner with the aid of a pyrometer at the sample end face. The control temperature is not to exceed the solidus temperature of the examined sample material in order to prevent melting of the sample.

In another embodiment, the temperature at the end face can also be measured in a contacting manner, e.g., with a thermocouple. This is not associated with undesired heat flows, as the temperature at the other end face is controlled and thus all heat transmission modes are already included.

The unheated sample end is located in a closed cooling circuit, through which a cooling liquid flows.

Using thermal detectors (e.g., thermocouples or resistance thermometers), the temperature of the cooling liquid in front of and behind the sample is measured continuously. A swirler positioned behind the sample and in front of the thermal detector forms a fluidic obstacle for the flowing cooling liquid and produces turbulent flow, whereby the cooling liquid after the sample is mixed and a homogeneous temperature distribution is ensured at the location of the thermal detector.

At any time, the amount of flowing cooling liquid is measured with the aid of a flow meter.

In the event that, in the method described here, the flow rate of the cooling liquid is important for the further determination of the thermal material properties, it is possible to vary the flow rate with a control valve integrated in the cooling circuit.

For continuous, non-contact characterization of the thermal state of the sample, the device in accordance with the invention further comprises an infrared camera, with which both individual pixels, individual lines or selected surfaces can be captured with regard to the intensity of the infrared radiation and therefore the temperature of the sample can be ascertained with a chronologically and locally variable resolution (e.g., from a few milliseconds to minutes as well as from 50 μm to millimeters). In order to avoid errors when measuring the radiation intensity on reflective samples, the surfaces are either blackened or filters are used. The accuracy of the temperature measurement is ensured by means of a certified calibration.

In a further embodiment, the pyrometer can be dispensed with for temperature control and instead a defined array of pixels (e.g., 3×3 pixels or a line transverse to the direction of heat flow) from a thermogram of the sample created with the aid of the aforementioned infrared camera can be used for control purposes.

In order to prevent lateral heat losses from the sample, the sample is enclosed in a thermal isolator which has an aperture in the direction of the infrared camera used. This aperture is closed with a material transparent to infrared radiation of a defined wavelength.

In another embodiment of the device in accordance with the invention, the thermal isolator consists of a material transparent to infrared radiation of a defined wavelength and completely encloses the sample material.

In a further embodiment, the isolation material is additionally silvered on the inner side in order to minimize thermal radiation losses.

Depending upon the embodiment, the sample is present in a rod-shaped geometry and has a planar surface in the direction of the infrared camera. In order to ensure constant emissivity over the entire temperature range, the sample is blackened in the measurement region. The optical radiation properties defined in this manner ensure precise temperature measurement irrespective of the wavelength range used and the sample material to be examined. Furthermore, the blackening of the sample in the case of heating by laser favors the absorption into the material to be examined, thus enabling controlled heating of one side of the sample.

The device in accordance with the invention is used to determine the thermal diffusivity during heating and cooling with a suitable heating or cooling rate and the resulting transient states. Thermal conductivity is determined in the thermally steady state of the sample within the same measuring cycle. If both thermal diffusivity and thermal conductivity have been ascertained, the specific heat capacity is calculated with the aid of these two material properties. The major advantage over the prior art resides in the fact that the thermal material properties (thermal diffusivity, thermal conductivity and specific heat capacity) of the examined sample material can be determined directly and over a large temperature range simultaneously with only one measuring device and within one measuring cycle.

From the temperature-dependent progression of the thermal properties of an examined sample material which are determined with the device in accordance with the invention, conclusions can be drawn in respect of further material-specific properties. For example, material-specific and temperature-specific phase transitions such as order transitions, allotropic or polymorphic transformations and/or magnetic transformations at the Curie temperature T_{S} and/or Néel temperature T_{N} can be detected on the basis of monotonic, sudden and/or abrupt changes in the progression of thermal diffusivity and/or thermal conductivity and/or specific heat capacity. Therefore, a targeted further examination of these phenomena is substantially simplified. Furthermore, the device in accordance with the invention renders it possible to characterize samples with different phases within the sample region detected by the infrared camera. It is also possible to examine single-phase samples with concentration differences generated in the axial direction and to determine the concentration-dependent change in thermal properties on the basis of the change in thermal properties in comparison with samples with a uniform concentration.

In a further embodiment of the device in accordance with the invention, it is possible to examine rod-shaped samples with a low height. For this purpose, the sample is heated on one side with a susceptor, as described above. On the opposite side, the sample is cooled with a cylinder extending into the cooling liquid. With knowledge of the heat transfer coefficient between the sample and the cooling cylinder, the size of the contact surface and the temperature difference between the sample and the cooling cylinder in the region of the contact surface, it is possible to determine the heat flow in the sample and to carry out the evaluation with regard to the thermal properties on the basis of the measured temperature profiles in the manner in accordance with the invention.

The device and the method will be explained hereinafter with reference to exemplified embodiments and with the aid of FIGS. 1 to 7. The same reference signs are used for parts which are identical or have an identical effect.

In the figures:

FIG. 1 shows a schematic overall view of the device for simultaneously determining temperature-dependent thermal conductivity, thermal diffusivity and specific heat capacity of a sample to be examined,

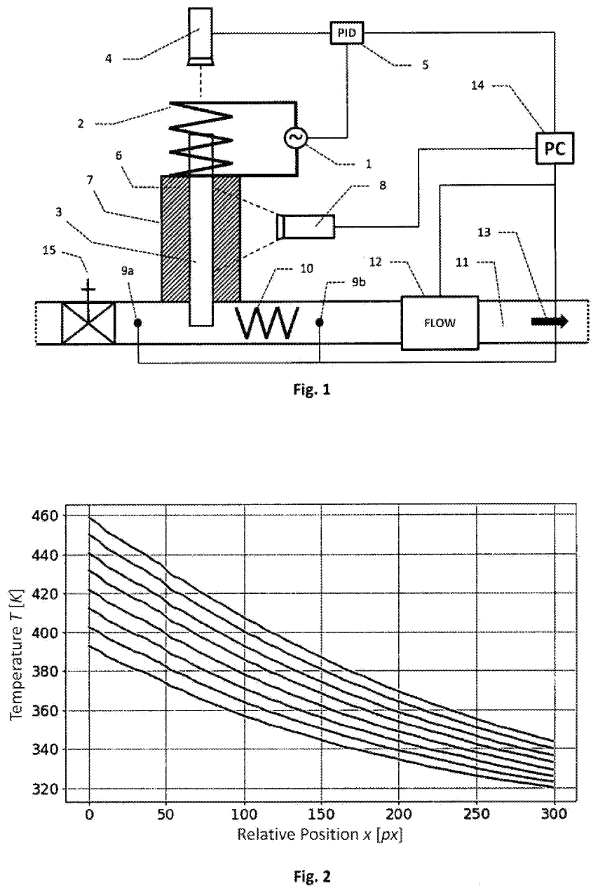

FIG. 2 shows exemplary transient temperature profiles during rapid heating of a sample end,

FIG. 3 shows exemplary temperature profiles of a sample in the steady state for different constant, controlled temperatures (T_{py}) at the heated sample end,

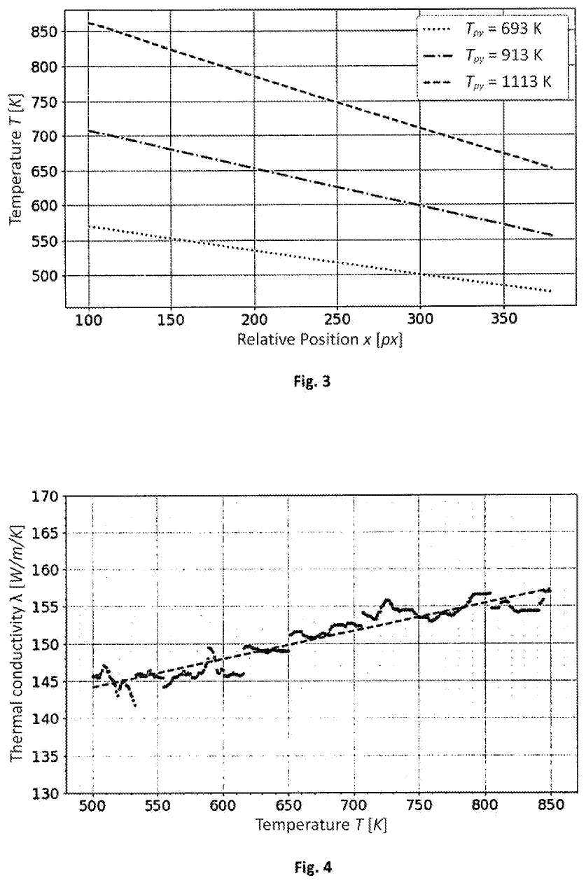

FIG. 4 shows the temperature-dependent progression of thermal conductivity over a large temperature range in linearized form (as a first-order polynomial),

FIG. 5 shows the progression of thermal diffusivity determined experimentally from the transient states in linearized form (as a first-order polynomial),

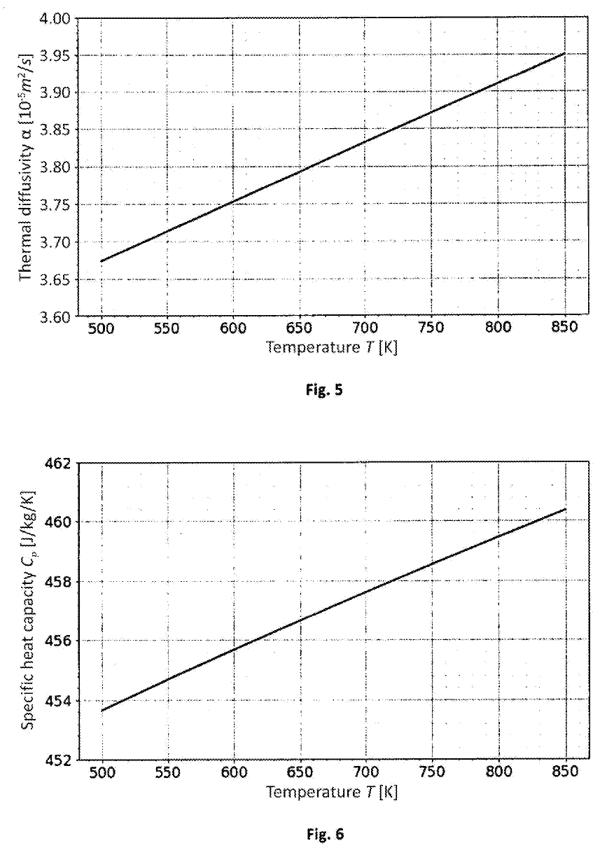

FIG. 6 shows the temperature-dependent progression of specific heat capacity determined from the data sets in FIG. 4 and FIG. 5,

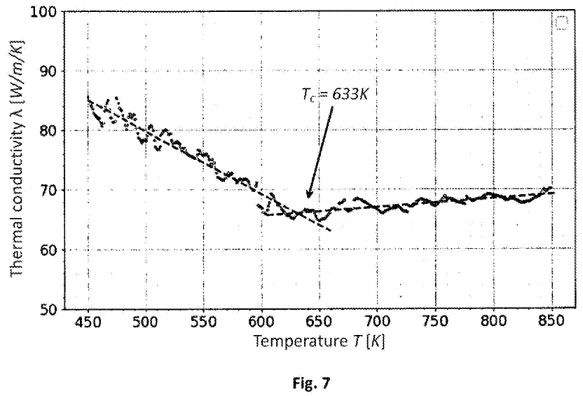

FIG. 7 shows by way of example the transition (in this case Curie temperature T_{c}) detected from the temperature-dependent progression of thermal conductivity for pure nickel (Ni).

### DETERMINING THERMAL DIFFUSIVITY

In order to transfer the sample 3 into different transient states which are characterized by temperature gradients ∂T/∂x which differ at any time along the sample axis, in one embodiment of the device in accordance with the invention the heating of the upper sample end to a temperature T_{py} is achieved by means of PID-controlled power control of an induction furnace 1.

By reason of the rotationally symmetrical geometry of the induction coils 2 used in practice it is expedient also to provide the sample 3 to be examined with a rotationally symmetrical geometry. Since the production of rotationally symmetrical cylindrical rods is possible with little machine outlay, in one embodiment of the device in accordance with the invention the sample 3 to be examined should have a cylindrical geometry with a length between 20 and 80 mm and a diameter between 4 and 10 mm, but in particular a length of 50 to 60 mm and a diameter of 8 mm.

The upper end of the cylindrical sample 3 is positioned centrally in the induction coil 2. The primarily electrically conductive sample material inductively couples to the alternating field of the induction coil 2. As a result of this, the sample 3 is heated in a contactless manner owing to the induction currents generated in the material, and initially passes through a thermally transient state. Electrically semi-conductive or non-conductive materials can be heated with the aid of a susceptor and also using the induction coil 2.

In order to prevent the sample material melting inside the induction coil 2, the sample end is located over half the coil height and the maximum temperature of the sample 3 at the upper end face T_{py} is measured continuously and in a contactless manner using a pyrometer 4. The measured temperature serves as an input for a PID controller 5 which controls the power of the induction furnace 1.

The heating output is varied during heating (e.g., by briefly switching the induction furnace 1 on or off and/or by sinusoidally modulated heating output of the induction furnace 1, which varies in amplitude). By means of the one-sided heating and varied heating output, the sample 3 is put into a thermally transient state at any time t and at any location x and, in contrast to the LFM, it is possible, within an extremely short time, to determine the temperature-dependent thermal diffusivity α(T) within a large temperature range.

The transient states are continually recorded using an infrared camera 8. Undesired lateral heat losses are prevented or greatly reduced by a thermal isolator 6. Furthermore, thermal radiation losses are minimized by silvering 7 applied to the thermal isolator 6. FIG. 2 shows different thermally transient states along the sample axis, which can be achieved during heating of a sample 3 (in this case Cu₇₀Zn₃₀). The illustrated temperature profiles are the arithmetic mean of a plurality of lines of the detector which extend in parallel with each other and which all lie within the just prepared surface along the sample axis.

The thermal diffusivity always results from two temperature profiles as illustrated in FIG. 2, and is determined multiple times according to the response time of the infrared camera 8 used in one embodiment of the invention. The capture rate of the infrared camera 8 during the analysis of the transient states amounts to 5 ms to I s, but more particularly 20 ms. High capture rates permit multiple determinations and subsequent averaging of the determined thermal diffusivities and increase the achievable accuracy of the method compared to the single determination.

In one modification of the method, in addition to the temperature profiles along the sample axis documented during heating, the transient thermal states are evaluated during the cooling process. The temperature profiles which can be evaluated are thereby multiplied, whereby the accuracy of the measurement of the thermal diffusivity is further increased.

The basis of the method described herein for determining thermal diffusivity forms an inverse numerical method, in which, starting from an initially freely selected starting value for the thermal diffusivity a, the following homogeneous thermal conductivity equation is iteratively solved.

$$\frac{\partial T}{\partial t} = \frac{\partial }{\partial x} ( α \cdot \frac{\partial T}{\partial x} ) \\ with \\ α = \frac{λ}{ρ \cdot {c}_{p}}$$

The temperature-dependent thermal diffusivity is described by a polynomial of the nth order (n=1, 2, 3, . . . , but in particular for a moderate temperature range n=1). Once the thermal conductivity equation has been solved using the polynomial in an iteration step, the calculated temperature profiles are compared with the temperature profiles measured using the infrared camera. If calculated and measured profiles differ from each other, the parameters of the polynomial used to calculate the temperature-dependent thermal diffusivity are adapted and the thermal conductivity equation is solved again. The parameters are adapted using smallest error square methods. This procedure is repeated until the calculated temperature profiles match those measured by the infrared camera 8 to the best possible degree. The evaluation of the whole temperature profile therefore results in a temperature-dependent progression of the thermal diffusivity, as shown by way of example in FIG. 5.

Determining Thermal Conductivity

When the temperature distribution along the sample 3 no longer changes, the sample 3 is in the thermally steady state and the temperature-dependent thermal conductivity λ(T) of the sample material used can be determined using the heat flow \dot{Q}/A through the sample 3 and the evaluation of the temperature profile ∂T/∂x along the sample 3.

In order to achieve the thermally steady state, the temperature T_{py} at the heated sample end is measured using a pyrometer 4 and kept constant by a suitable PID controller 5. The temperature of the cooling liquid (measured by thermal sensor 9a) is kept constant by means of a thermostat. At the cool sample end, the cooling liquid flows around the sample 3, the temperature of said liquid being raised by the amount of heat given off by the sample. In the steady state, the amount of heat \dot{Q} given off to the cooling liquid per unit of time no longer changes and the temperature change of the cooling liquid (Δ_{f}Tₗ) determined as the difference of the temperature of the thermal sensor behind the sample 9b and the temperature of the thermal sensor in front of the sample 9a is constant.

In the thermally steady state, the sample 3 has the same amount of heat flowing through it along the sample axis in each cross-section A. The heat which is supplied to the heated sample end by means of the induction furnace 1 flows in the direction of the cooled end of the sample, which is located in the closed cooling circuit 11. As the cooling liquid is flowing past the sample, the heat is fully transmitted to the cooling liquid. A swirler 10 then mixes the cooling liquid in order to ensure a homogeneous temperature distribution in the cooling liquid before the temperature increase is quantified by means of two thermal sensors 9. The temperature per cross-sectional surface area of the sample no longer changes and remains constant.

The heat flow \dot{Q}/A is defined as the amount of heat Q transmitted perpendicularly to the sample cross-sectional surface area A per unit of time t.

In the steady state, the relationship between the heat flow and the temperature profile ∂T/∂x resulting therefrom within the examined sample is described using the Fourier equation.

$$\frac{\dot{Q}}{A} = λ \cdot \frac{\partial T}{\partial x}$$

In an isotropic medium, heat flow and temperature gradient are directly proportional to one another. The proportionality factor is the thermal conductivity λ.

By reason of the thermal isolation 6 lateral heat losses are negligible and the one-dimensionality of the heat flow along the sample axis is ensured.

The quantification of the amount of heat which flows through each sample cross-section and is given off to the cooling liquid is effected with knowledge of the temperatures of the cooling liquid in front of (measured by the thermal sensor 9a) and behind the sample (measured by thermal sensor 9b) and of the flow rate of the cooling liquid per time interval m_{f}/₁/Δt

With the aid of the infrared camera 8 integrated in one embodiment of the invention, the temperature distribution of the sample 3 to be examined is recorded in the steady state along the just prepared surface.

From the temperature distribution measured along the sample axis, individual temperature profiles, which are in parallel with each other and extend in the axial direction, are extracted and then averaged. With knowledge of the local resolution of the infrared camera 8 used, the captured pixels are converted into metric lengths. The averaged one-dimensional temperature distribution describes with high resolution the temperature gradient ∂T/∂x [K/m] along the sample axis. By way of example FIG. 3 illustrates temperature distributions determined by means of this method. For different control temperatures T_{py} in each case the achievement of the steady state has been awaited and the temperature distribution along the sample axis has been determined.

The unheated sample end is integrated into the closed cooling circuit 11. In this case, cooling liquid flows around the sample 3 in flow direction 13. By reason of the lateral thermal isolation 6, the entire amount of heat generated at the upper sample end is given off to the cooling liquid.

The temperature of the cooling liquid changes by reason of the absorbed heat quantity Q. The temperature increase ΔT_{fl} depends upon the specific heat capacity of the cooling liquid c_{p;fl}, the amount of heat given off per time interval \dot{Q} and the flow rate of the cooling liquid per time interval m_{f}/₁/Δt. Therefore, the amount of heat given off to the cooling liquid per time interval \dot{Q} can be determined with the following equation:

$$\dot{Q} = \frac{Δ Q}{Δ t} = {c}_{p ; fl} \cdot \frac{{m}_{fl}}{Δ t} \cdot {Δ T}_{fl}$$

The flow rate of the cooling liquid per time interval m_{f}/₁/Δt is determined continuously with a flow meter 12. Furthermore, a control valve 15 for setting the flow rate is integrated into the cooling circuit 11. This ensures that a sufficiently large temperature increase Δ_{f}T is achieved between the thermal sensors 9a and 9b.

The heat flow \dot{Q}/A along the sample axis can be calculated with knowledge of the sample cross-sectional surface area A. With the aid of the measured temperature gradient ∂T/∂x, the temperature-dependent thermal conductivity λ(T) is calculated by transposing the Fourier equation. In order to increase the accuracy of the method, thermal conductivity is determined multiple times at one temperature by selecting the temperatures T_{py} set at the heated end such that the different temperature ranges of the respective steady states overlap. The temperature-dependent thermal conductivities λ(T) determined with the aid of the method described in this case are illustrated by by way of example in FIG. 4. In addition, a linearly adapted curve is illustrated over the complete temperature range examined.

Determining Specific Heat Capacity

After determining temperature-dependent diffusivity α(T) from the transient states during heating or cooling and determining thermal conductivity λ(T) from steady states, the temperature-dependent specific heat capacity cₚ (T) of the sample is calculated with the aid of the following relationship:

$${c}_{p} ( T ) = \frac{λ ( T )}{ρ \cdot α ( T )}$$

FIG. 6 shows by way of example the temperature-dependent progression of specific heat capacity of

$${Cu}_{70} {Zn}_{30} [ p = 8.65 \cdot \frac{{10}^{3} kg}{{m}^{3}} ) ,$$

as calculated with the aid of the temperature-dependent thermal diffusivity, determined in accordance with the invention, from FIG. 5 and the temperature-dependent thermal conductivities from FIG. 4.

Derived Properties

Further temperature-dependent properties of the examined sample material can be derived on the basis of the temperature-dependent progression of thermal diffusivity α(T), thermal conductivity λ(T) and/or specific heat capacity cₚ(T).

Abrupt and/or sudden changes and/or change in monotonicity and/or a change in the increase in the temperature-dependent progression of thermal diffusivity α(T), thermal conductivity λ(T) and/or specific thermal conductivity cₚ(T) are indications of phase transitions such as order transitions, allotropic or polymorphic transformations and/or magnetic transformations at the Curie temperature T_{c} and/or Néel temperature T_{N} and/or for the existence of different phases along the sample axis and/or concentration differences extending in the axial direction within a phase.

FIG. 7 shows by way of example the change in the increase in the progression of thermal conductivity of nickel at a temperature of T=630 . . . 640 K. In accordance with the literature, pure nickel undergoes a reversible transition at the Curie temperature of T_{c}=633 K. Below the Curie temperature, pure nickel is ferromagnetic, and is paramagnetic above it.

The device and method for determining temperature-dependent thermal diffusivity, thermal conductivity and specific heat capacity described in this case can thus also be used for analyzing material-specific transitions, such as order transitions, allotropic or polymorphic transformations and/or magnetic transformations at the Curie temperature T_{c} and/or Néel temperature T_{N} and/or for analyzing different phases which occur along the sample axis and/or for evaluating concentration differences, extending in the axial direction, within a phase.

### LIST OF REFERENCE SIGNS

1 induction furnace
2 induction coil
3 sample
4 pyrometer
5 PID controller
6 thermal isolation
7 silvering
8 infrared camera
9 thermal sensor
10 swirler
11 closed cooling circuit with fluid cooling medium
12 flow meter
13 flow direction
14 computer
15 control valve

1. A method for simultaneously determining thermal conductivity, thermal diffusivity and specific heat capacity, comprising the steps of
locally heating a sample (3) to be examined at a sample end,
performing non-contact temperature measurement along the sample (3),
measuring the temperature change in a cooling liquid flowing around the other sample end,
in order to measure transient and steady thermal states of the sample (3) and determine the thermal diffusivity from the transient thermal states and to determine the thermal conductivity from the steady state and then calculate the temperature-dependent specific heat capacity.

2. The method according to claim 1, characterized in that the thermal states are produced by heating an electrically conductive sample (3) on one side by means of a controlled power output of an induction furnace (1).

3. The method according to claim 1, characterized in that the thermal states are produced by heating an electrically conductive and/or semi-conductive and/or non-conductive sample (3) on one side by means of a controlled power output of an induction furnace (1) with the aid of a susceptor.

4. The method according to claim 1, characterized in that the thermal states are produced by heating an electrically conductive and/or semi-conductive and/or non-conductive sample (3) on one side by means of a controlled power output by a laser.

5. The method according to claim 1, characterized in that the thermal states are produced by heating an electrically conductive and/or semi-conductive and/or non-conductive sample (3) on one side by means of a controlled power output by a resistance-heated heater.

6. The method according to claim 1, characterized in that the thermal states in short samples (3), in particular samples smaller than 20 mm, are produced by heating on one side and cooling on both sides using a cooling body extending into the cooling liquid.

7. The method according to claim 1, characterized in that the temperature at the heated sample end is set by a PID controller (5).

8. The method according to claim 1, characterized in that the temperature-dependent thermal diffusivity is calculated by an inverse numerical method as a polynomial of the nth order, where n is an integer, preferably n=1.

9. The method according to claim 1, characterized in that the flow rate of the cooling liquid is controllable and/or is determined continuously with a flow meter (12).

10. A device for performing a method according to claim 1, comprising:
an induction furnace (1) and/or an induction furnace (1) in conjunction with a susceptor and/or a laser and/or a resistance-heated heater for heating the sample (3),
a pyrometer (4) and/or an infrared camera for determining the temperature at the heated sample end and/or for relaying to a controller,
a PID controller (5) for setting defined heating and/or cooling rates and/or a constant temperature at the heated sample end,
a thermal isolation (6) for avoiding lateral heat losses,
an infrared camera (8) for measuring the temperature progressions along the sample (3) in thermally transient and/or steady states,
one, two or more thermocouples, resistance thermometers and/or other thermal detectors for determining the coolant temperature,
a swirler (10) for producing a homogeneous temperature of the cooling liquid behind the sample (3),
a flow meter (12) for determining the flow rate of the coolant, and
a control valve (15) for setting the flow rate of the cooling liquid.
